(19) United States
(12) Patent Application Publication
(10) Pub. No.: US 2010/0280469 A1
(43) Pub. Date: Nov. 4, 2010

(54) WOUND FILLERS

(75) Inventors: Kristian Hall, Hull (GB); Edward Hartwell, York (GB); Derek Nicolini, Brough (GB)

Correspondence Address: KNOBBE MARTENS OLSON & BEAR LLP, 2040 MAIN STREET, FOURTEENTH FLOOR, IRVINE, CA, 92614, US

(73) Assignee: Smith & Nephew Plc, London (GB)

(21) Appl. No.: 12/746,508

(22) PCT Filed: Nov. 26, 2008
(86) PCT No.: PCT/GB2008/051122

§ 371 Date: Jun. 4, 2010

(30) Foreign Application Priority Data
Dec. 6, 2007 (GB) 0723852.0

(51) Int. Cl. A61M 1/00

(52) U.S. Cl. 604/319

(57) ABSTRACT

A wound filling device for use in apparatus for the application of topical negative pressure therapy to a site on the body of a mammal is described, the device comprising: an inflatable bag member having at least one fluid carrying conduit operably connected thereto to inflate/deflate said bag member; a separate textured covering sock member at least partially covering the inflatable bag member. Apparatus embodying the wound filling device is also described.

The present invention relates to apparatus and devices for use in the treatment of wounds by topical negative pressure (TNP) therapy.

TNP therapy often involves the provision within a dressing over wound to which a negative or a positive pressure is applied a bag member (sometimes also referred to as a bladder) which may be used to at least partially fill the volume of a wound, for example. Furthermore, fluctuating pressure or pressure cycling may be applied to the bag member in order to work the tissue in and around the wound region for therapeutic reasons, for example.

In our co-pending International patent application, WO 2004/037334, apparatus, a wound dressing and a method for aspirating, irrigating and cleansing wounds are described. In very general terms, this invention describes the treatment of a wound by the application of topical negative pressure (TNP) therapy for aspirating the wound together with the further provision of additional fluid for irrigating and/or cleansing the wound, which fluid, comprising both wound exudates and irrigation fluid, is then drawn off by the aspiration means and circulated through means for separating the beneficial materials therein from deleterious materials. The materials which are beneficial to wound healing are recirculated through the wound dressing and those materials deleterious to wound healing are discarded to a waste collection bag or vessel.

In our co-pending International patent application, WO 2005/04670, apparatus, a wound dressing and a method for cleansing a wound using aspiration, irrigation and cleansing wounds are described. Again, in very general terms, the invention described in this document utilises similar apparatus to that in WO 2004/037334 with regard to the aspiration, irrigation and cleansing of the wound, however, it further includes the important additional step of providing heating means to control the temperature of that beneficial material being returned to the wound site/dressing so that it is at an optimum temperature, for example, to have the most efficacious therapeutic effect on the wound.

In our co-pending International patent application, WO 2005/105180, apparatus and a method for the aspiration, irrigation and/or cleansing of wounds are described. Again, in very general terms, this document describes similar apparatus to the two previously mentioned documents hereinabove but with the additional step of providing means for the supply and application of physiologically active agents to the wound site/dressing to promote wound healing.

The content of the above references is included herein by reference.

All of the wounds to which the above documents are addressed may require the provision of a bag member or bladder within a wound dressing and those currently available may be unsuitable. Indeed, our co-pending International patent application WO 2004/037334 referred to above shows at FIGS. 13A and 20 embodiments employing pre-formed inflatable bladders used in apparatus for wound therapy.

Other reasons why inflatable bladder-type wound fillers may be used is to apply an uneven or textured or otherwise non-smooth surface possessed by the bladder surface directly onto a wound surface/interface. The textured surface assists in “working” the wound surface thereby enhancing the therapeutic effect of pressure cycling. A textured surface also provides a plurality of fluid flow channels at the interface between the wound filler and the wound surface to assist in a more even (negative) pressure distribution over the wound surface and a more rapid and even drainage of fluids such as wound exudates, for example, away from the wound region towards an aspiration conduit generally forming part of a TNP therapy apparatus.

Examples of known inflatable wound fillers are described in WO-A-2005/082435 and GB-A-2378392.

In WO-A-2005/082435, an inflatable bag or bladder-type wound filling member is used to fill the wound cavity. The bladder member is described as having two layers, however, the two layers appear to be in the form of an integral structure. A problem with this type of structure is that it is difficult and costly to manufacture. The structure also makes the bladder relatively rigid and stiff and inflexible against a wound surface possibly leading to discomfort and trauma.

GB-A-2378392 describes a wound irrigation and/or suction device comprising an inflatable bladder or pouch having a plurality of flexible conduits provided within and running through the bladder interior, the conduits having outlet/inlet apertures on the bladder surface. The conduits are fluid transfer conduits to either supply fluid to the wound or to conduct fluid away from the wound or both. However, a problem with this particular construction is that it is extremely complex and costly to manufacture.

It is an objective of the present invention to remove or at least mitigate some of the disadvantages of known device structures.

According to a first aspect of the present invention there is provided a wound filling device for use in apparatus for the application of topical negative pressure therapy to a site on the body of a mammal, the device comprising: an inflatable bag member having at least one fluid carrying conduit operably connected thereto to inflate/deflate said bag member; a separate textured covering sock member at least partially covering the inflatable bag member.

The wound filling device as described above may form part of an apparatus for the application of TNP therapy to a wound, for example, on the body of a human being.

Such apparatus may also generally include an aspiration conduit connected to aspiration means such as a vacuum pump, for example, for aspirating a wound cavity defined beneath a sealing membrane or drape covering adhered to sound skin or flesh surrounding the wound and beneath which the inflatable bag member and sock member are enclosed. The at least one conduit operably connected to the inflatable bag for inflating/deflating may also pass through or under the sealing membrane or drape but in any event be sealed thereto in accordance with well known TNP dressing structures. Provision of negative pressure may be achieved by any means or methods known in the art to evacuate the wound cavity region.

The inflatable bag member may be operably connected to a suitable means for inflation thereof via the at least one conduit. Such means may include well known types of pump able to pump inflation fluid, such as air for example, into the bag and also perhaps to suck fluid out so as to either maintain a constant pressure therein or to apply a pulsating pressure regime within the bag so as to work the wound surface to achieve beneficial therapeutic effects on the wound.

Whilst the option of positive pressure inflation of the bag member is described above there is also the option of allowing the bag member to self-inflate by leaving the conduit connected thereto open to atmosphere. As the wound cavity surrounding the bag member is evacuated by a vacuum pump, for example, the bag will tend to inflate by drawing air into it purely due to the pressure differential between the wound cavity and the bag interior as the bag surfaces are drawn towards the wound surface on one side and to the inner surface of the sealing drape membrane on the other side.

The wound cavity formed beneath the sealing membrane or drape may also have a further conduit provided thereto to supply an air bleed into the wound cavity so as to maintain a constant negative pressure therein to avoid undue discomfort to a patient due to excessive negative pressures being applied where the sealing membrane is particularly effective in sealing the wound cavity. Such a bleed conduit will also promote continuous aspiration of the wound cavity. Such an additional conduit may also serve the dual purpose of be used to measure pressure at the wound when a pressure transducer is connected to it distal to the wound.

A further conduit may be provided having access to the wound cavity region for the provision or application of an irrigant and/or medicaments to the wound site.

The inflatable bag member and the sock member may be mutually slideable relative to each other. Thus, as the bag member is inflated and deflated in a predetermined pressure pulsing regime, for example, the outer surface of the bag member and the inner surface of the sock member may slide against one another. This prevents the textured surface of the sock member from being forced to slide against the wound surface and causing possible trauma thereto and potentially damaging newly formed granulation tissue and thus, the outer textured surface of the sock member may apply pressure pulses substantially normally to the wound surface.

By providing the inflatable bag member and the textured sock member as two separate items made by known economic manufacturing techniques several important advantages are gained. Firstly, the inflatable bag member may be made from inherently soft and flexible thin sheet material which remains so even when having folds and convolutions when inside the wound cavity thereby imposing a minimum of unnecessary stress and trauma on the wound surface compared with known multi-layer bag constructions. The material from which the inflatable bag member may be made may be relatively very thin since the pressures which it has to withstand on inflation are relatively low. Secondly, by having a separate sock member over the inflatable bag member, the sock member surface texturing may be tailored to the requirements of the wound surface and have a suitable sock/wound interface design adapted to the wound type.

The sock member may be made from two sheets of material having substantially coterminous outer shapes, for example, and be welded together at their outer peripheries. One sheet may have an aperture, for example, in the centre through which the inflatable bag member is to be inserted, and also about which aperture the sock member may be turned inside out so as to contain the welded periphery on the inside of the sock member out of contact with the wound surface.

The inflatable bag member may be made from thin, flexible, substantially impervious plastics sheet material such as EVA, PU, PP, PE, silicone and any other suitable flexible plastics materials, for example, in similar manner to the sock member in that after welding it may be turned inside out to contain the weld on the inside. However, this is not so important as with the sock member as the inflatable bag member is contained within the sock member so preventing the welded periphery from contacting the wound.

Thus, by having separate bag and sock members each component may be made having properties which are optimised for its function and not compromised by having to fulfil two separate roles.

In one embodiment of an inflatable bag member and sock member the sheets from which they are made may be circular, for example.

The sock member may be moulded as an entity for subsequent combination with the inflatable bag member.

The conduit for inflating/deflating the bag member may be sealed to the bag member where it passes into the interior thereof. However, in a preferred embodiment of the present invention the inflatable bag member is provided with an aperture to which is affixed, by welding for example, a port member which may provide fluid access to the inflatable bag member and advantageously, fluid access to and from the wound cavity region. Such a port member may have provision for joining suitable fluid conduits such as flexible plastics materials conduits. The port member may also advantageously be provided with a suitable shroud member which serves the dual purpose of providing a smooth rounded surface which does not cause discomfort to a patient should it be lain upon and also importantly, maintaining a free flow of fluids from the wound site without the various ports being occluded or blocked by the overlying sealing drape membrane. The port member and shroud provides the advantage of having all of the conduits grouped together and being able to handle them as one which makes application of the apparatus to a wound quicker and more efficient and improving the reliability of the TNP apparatus.

In a preferred embodiment of the present invention, when in use, access of the various conduits to the wound cavity region below the sealing drape may be provided by a dressing and grommet combination as described in our co-pending GB patent application 0712735.0 and PCT/US 2007/074374 of common ownership herewith.

The construction of the device according to the present invention by using a soft and flexible sock material allows easier dressing removal since the sock may be made from material substantially non-adherent to the wound surface thus causing less distress and trauma to the patient.

The separate sock member of the device according to the present invention promotes a reduction in tissue growth tending to close the wound and leave a cavity. The sock member effectively forms an analogous member to wound fillers in conventional TNP therapy of preventing wound overgrowth and minimising the formation of pockets within the tissue and encouraging the wound to heal by secondary intent or from the wound base upwards.

The textured surface of the sock member may primarily be present to provide a plurality of channels over the surface of the bag/sock combination so as to provide an even pressure distribution over the whole surface area of the wound and also to provide by the same plurality of channels efficient and rapid drainage of wound exudate fluids therefrom.

In one embodiment of a sock member according to the present invention the textured surface may comprise an array of hexagonal indentations or depressions in the sock material surface effectively producing a three-dimensional surface structure, each indentation or depression having a perforation at its centre to allow fluid flow, both gaseous and liquid, over both surfaces of the sock member, i.e. between the wound surface and sock surface and between the inflatable bag surface and sock surface. A suitable material from which the sock member may be made may be a vacuum formed plastics sheet material such as, for example, EVA film which is soft and non-adherent to a wound surface.

The sock member may have any desired surface topography and formations which are conducive to wound surface therapy.

Alternative materials from which the separate sock member may be formed may comprise woven materials, non-woven fibrous sheet, foams, electrospun nano fibres from a wide variety of different materials such as EVA, PU, PP, PE, silicone, carbomethoxy cellulose, polyacrylate, for example.

Bio-degradable materials such as collagen, oxised cellulose, chitosan, polyglycolic acid and the like may be used, for example.

In one embodiment of the present invention the inflatable bag member, port and shroud members, conduits, an appropriate dressing and grommet member for attachment to a patient and sock member may be supplied as one integrated unit which merely needs to be applied to wound and connected to suitable sources of vacuum and inflation fluid to be put into effect as TNP therapy apparatus.

According to a second aspect of the present invention there is provided apparatus for the application of topical negative pressure therapy to a site on the body of a mammal and which apparatus embodies the wound filling device of the first aspect of the present invention.

In order that the present invention may be more fully understood, examples will now be described by way of illustration only with reference to the accompanying drawings, of which:

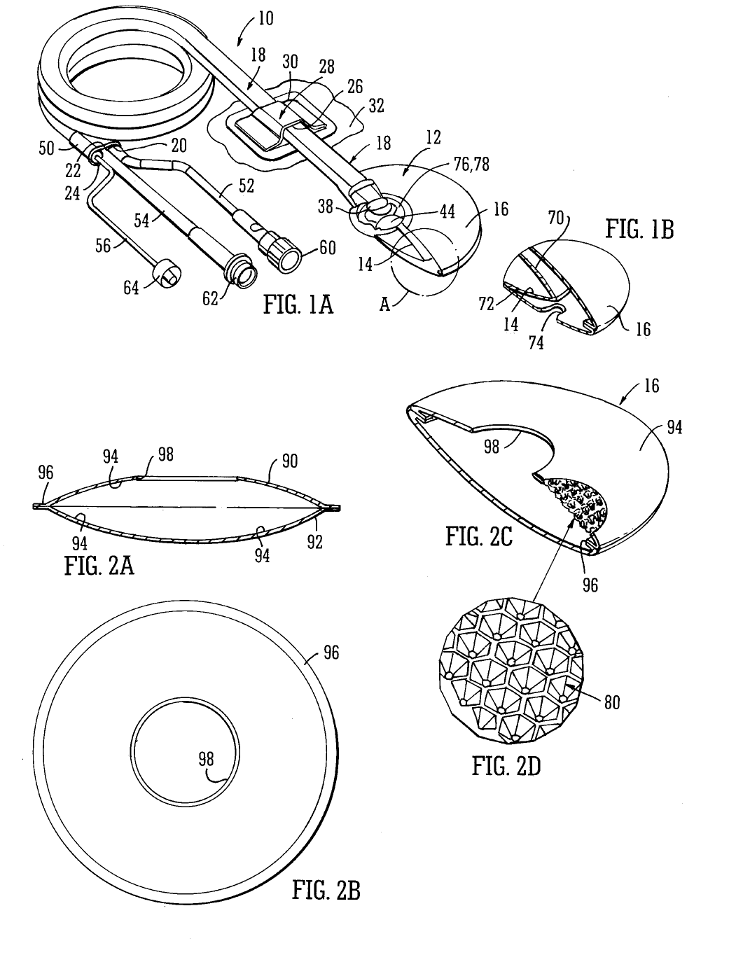

FIGS. 1A and 1B show a part sectioned perspective view of one construction of apparatus embodying a wound filling device according to the present invention;

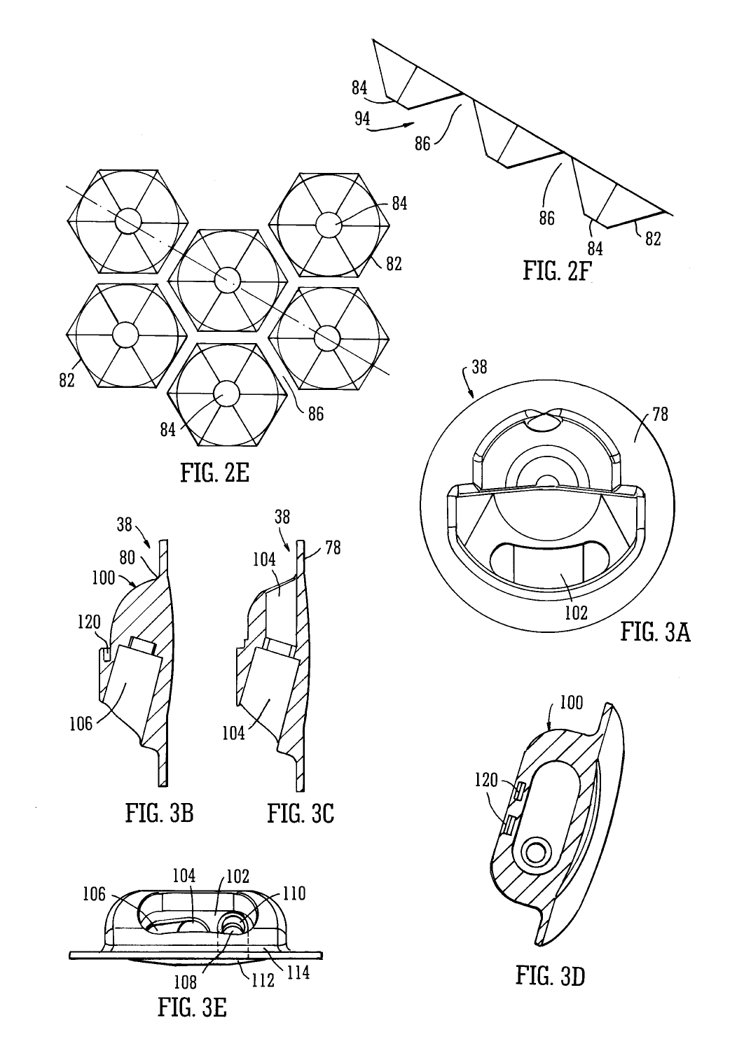

FIGS. 2A to 2F show various views of a sock member and its construction;

FIGS. 3A to 3E shows various sections and views of a port member for welding to an inflatable bag member;

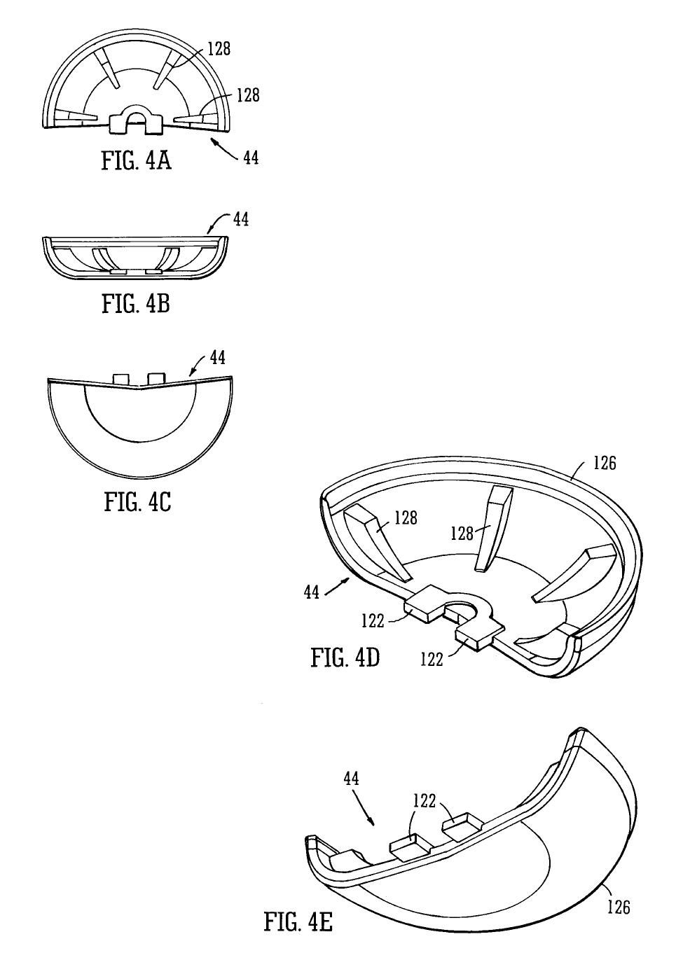

FIGS. 4A to 4E show various sections and views of a shroud member to co-operate with the port member of FIG. 3; FIG. 4A shows an underside plan view of a shroud member; FIG. 4B shows a side view in elevation of the shroud member of FIG. 4A; FIG. 4C shows a top plan view of the shroud member of FIG. 4A; FIG. 4D shows a perspective view of the underside of the shroud member of FIG. 4A; and, FIG. 4E shows a perspective view of the top of the shroud member of FIG. 4A; and

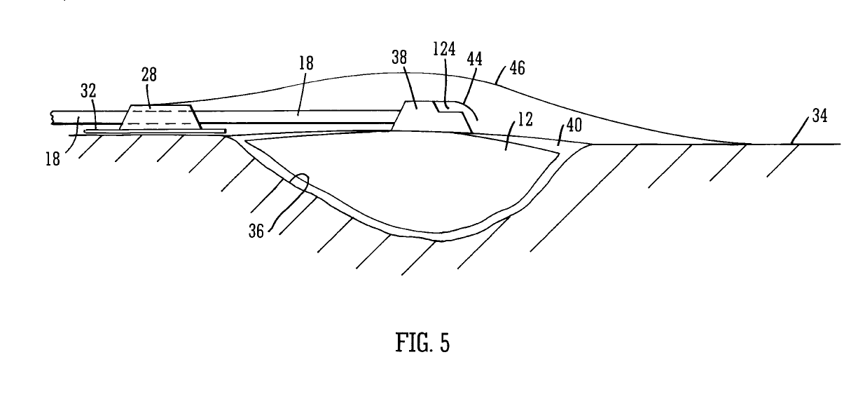

FIG. 5 which shows a schematic cross section of a wound having a wound filling device according to the present invention embodied in apparatus for the application of TNP therapy.

Referring now to the drawings and where the same features are denoted by common reference numerals.

FIGS. 1A and 1B show a perspective view of one embodiment of a device according to the present invention at 10 and FIG. 5 shows a schematic cross section of the device installed in a wound cavity. The device comprises a wound filling inflatable device 12 which is constituted by an inflatable bag member 14 and a sock member 16. The inflatable wound filling device 12 is shown in FIG. 1 part sectioned and a detail “A” is shown at a greater magnification in FIG. 1B. The device also comprises a multi-lumen conduit 18 (shown coiled up prior to use) having three separate lumens 20, 22, 24 therethrough. The conduit 18 passes through a hole 26 in a grommet member 28 which itself is adhered by a flange 30 around the periphery of the grommet member 28 to a dressing 32 which is shown in part only and which adheres the grommet 28 to a patients' skin 34 (FIG. 5) adjacent a wound 36 to which TNP therapy is to be applied. The arrangement is shown in a schematic cross section in FIG. 5. At the wound-end of the device the conduit 18 is attached to a port member 38 which has passages therethrough which connect the individual lumens 20, 22, 24: to the interior of the inflatable bag member 14; the wound cavity 40 (see FIG. 5) for the purpose of aspiration thereof; and to the wound cavity 40 for the purpose of providing an air-bleed thereto to maintain a constant negative pressure and/or to monitor pressure in the cavity 40, respectively. A shroud member 44 is provided which clips over the port member 38 for the purpose of preventing an overlying sealing drape membrane 46 from occluding the suction passage in the port member 38 (explained in detail with reference to FIG. 3 below). The drape membrane 46 seals around the periphery of the wound cavity 40 so as to substantially prevent large scale ingress of air as is well known in the TNP art. At the end of the conduit 18 remote from the wound, the conduit has a connector block 50 which connects the lumens 20, 22, 24 to separate conduits 52, 54, 56, respectively which are themselves provided with suitable connectors 60, 62, 64, respectively on their free ends for the purpose of connecting the lumens to a source of air (not shown) to inflate the inflatable bag member 14; to a vacuum source such as a vacuum pump (not shown) to aspirate the wound cavity 40; and, to a pressure relief valve and or pressure transducer (not shown) to provide an air bleed into the wound cavity to maintain a desired negative pressure in the wound cavity 40.

With regard to the dressing 32 and grommet 38 reference is made to our co pending GB patent application 0712735.0 and PCT/US 2007/074374 of common ownership herewith, the contents of which are included herein by reference, and which explain in detail the structure and constitution of these features but which are relatively incidental to the present invention and could be replaced with any suitable means of sealing the conduit 18 to the sealing drape 46 to prevent ingress of ambient air into the wound cavity from this particular source.

The wound filling device 12 comprises an inflatable bag member 14 having an outer covering sock member 16. The inflatable bag member 14 is made by welding two circular sheets 70, 72 of thin, flexible, impermeable plastics material together around their peripheries to form a weld bead 74. The upper sheet 70 has a central circular aperture 76 therein of a size suitable to be affixed to a thin flange 78 around the periphery 80 of the port member 38, the flange 78 and aperture 76 edge being either welded together or adhesively bonded so as to make the inflatable bag member 14 and the port member 38 an integral item. The sock member 16 is manufactured from a thin flexible plastics material such as EVA, for example and which has a vacuum-formed textured surface 80. The surface topography comprises an array of hexagons 82 having a central perforation 84 in each hexagon (although not all indentations may be perforated). The individual hexagons are separated by channels 86 as shown in FIGS. 2C to 2E. FIG. 2F shows the contour of the hexagonal indentations from a side view with upstanding indentations being adjacent the bag surface 16 in use. The sock member is made from two sheets 90, 92 of the plastics material and which sheets are placed together having the eventual wound contacting surface 94 to the inside as shown in FIG. 2A. The two sheets are welded together around their peripheries to leave a weld bead 96 around the outside, FIGS. 2A and 2B showing the sock member after welding but before turning inside out. The upper sheet 90 has a central aperture 98 therein and after welding together the sock so formed is turned inside out about the aperture 98 to form the sock as shown in FIG. 2C with weld bead 96 on the inside and the required textured surface 94 on the outside. The aperture 98 is used to insert the inflatable bag member 14 into the sock 16, the aperture 98 fitting around the port member welded flange 78. FIG. 2C shows the sock member 16 with a small portion of the area having the textured surface, however, this for illustration only and the whole upper and lower surface of the sock member 16 may be provided with the textured surface, though not necessarily the same textured surface on both sides. In the illustrated embodiment the hexagons 82 are 2.6 mm across the flats, the central perforations 84 are 0.6 mm in diameter and the depth of the hexagonal indentations from the planar surface is 1.05 mm. However, these dimensions are merely exemplary of one embodiment of a sock member according to the present invention and dimensions may vary according to the needs of a particular wound in other embodiments.

In this embodiment it is important that the sock member has a textured surface over substantially the whole area thereof as this maintains a continuous flow path for aspirated fluid to the port member 38 as described below.

Although the example of the wound filling device described above is stated to be made with circular sheets of material, it is of course merely exemplary and the inflatable bag 14 and sock member 16 may be made of any required shape and size to suit a particular wound shape and size or a range of wound shapes and sizes.

The port member 38 is moulded from a soft and conformable EVA material, for example, such that if lain upon by a patient no resulting trauma is caused by use of a hard material. The port member 38 comprises a main body portion 100 having passages for connection to lumens 20(52), 22(54), 24(56) in conduit 18 and the flange portion 78 which is welded to the periphery of aperture 76 of the inflatable bag member around the periphery 80 of the main body portion 100. The port member has a socket portion 102 corresponding to and co-operating with the outer shape of the conduit 18 such that the conduit may be plugged directly into the socket 102 and which effects simultaneous connections with the individual limens 20, 22, 24 therein. Lumen 22 connects with passage 104 which passes through the upper part of the main body portion 100 to communicate with the wound cavity 40 (see FIG. 5) for the purpose of evacuation thereof. Lumen 24 connects with passage 106 for the purpose of providing an air bleed to the wound cavity 40 and/or measuring the actual pressure applied to the wound cavity 40. Passages 104, 106 are connected internally in the port member 38 by virtue of a common connection of lumens 22, 24 in the socket portion 102. Lumen 20 of conduit 18 is connected individually to passage 108 by means of a raised spigot portion 110 in the base of the socket 102 and which seals with lumen 20 to provide a source of air to the interior of the inflatable bag member 14. Passage 108 communicates with the interior of the inflatable bag member by exiting through the base 112 of the port member as indicated by dashed lines 114 in FIG. 3E. The main body portion 100 is provided with small blind recesses 120 to receive tongues 122 of a shroud member 44 for the purpose of retaining the shroud member 44 to the port member 38. The shroud member 44 serves to provide an air space or cavity 124 between the port member 38 and the shroud member 44 above and around the exit of passage 104 to the outside of port member 38 so as to prevent it from being occluded by the overlying drape membrane 46 during aspiration (see FIG. 5). The shroud member 44 comprises a half-clam shell body portion 126 sufficiently rigid to withstand the negative pressure applied by the drape membrane 46 without occluding passage 104. The clam shell body portion 126 is reinforced against excessive distortion by ribs 128.

As will be realised by those skilled in the TNP art many of the features described in the above embodiment may be changed without departing from the invention which is defined in the claims appended hereto. For example the unitary conduit 18 may be changed for separate conduits and an appropriate grommet member 28 or the grommet may be dispensed with and the conduits sealed to the overlying drape as is known in the TNP art. Similarly, the port member may be adapted to co-operate with separate conduits and a shroud member appropriately modified so as to provide the essential requirement of being able to aspirate the wound cavity without hindrance. The form of the texturing of the sock surface and the material of which it is made may be varied insofar as the sock does not cause unnecessary trauma to the wound and is able to maintain an even pressure distribution over the whole of the wound surface area. These and many other modifications may be made without departing from the scope or spirit of the present invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A wound filling device for use in apparatus for the application of topical negative pressure therapy to a site on the body of a mammal, the device comprising: an inflatable bag member having at least one fluid carrying conduit operably connected thereto to inflate/deflate said bag member; a separate textured covering sock member at least partially covering the inflatable bag member.

2. A wound filling device according to claim 1 wherein the bag member is made from two sheets of material welded together at their outer peripheries.

3. A wound filling device according to claim 1 wherein the sock member is made from two sheets of material welded together at their outer peripheries.

4. A wound filling device according to claim 3 wherein the sock member has said weld contained within the interior of the sock.

5. A wound filling device according to claim 1 wherein the sock member is moulded.

6. A wound filling device according to claim 1 wherein the sock member has an aperture therein for the insertion of the bag member thereinto.

7. A wound filling device according to claim 1 wherein the bag member and the sock member are able to slide relative to each other during inflation or deflation of the bag member.

8. A wound filling device according to claim 1 wherein the sock member is made from thin textured sheet plastics material.

9. A wound filling device according to claim 1 wherein the texturing comprises a 3-dimensional pattern.

10. A wound filling device according to claim 8 wherein the 3-dimensional pattern provides aspiration channels for drainage of wound exudate between an outer surface of the bag member and an inner surface of the sock member.

11. A wound filling device according to claim 9 wherein the 3-dimensional pattern also provides aspiration channels for wound exudate between an outer surface of the sock member and the wound surface.

12. A wound filling device according to claim 7 wherein the texturing comprises an array of repeated indentations.

13. A wound filling device according to claim 1 wherein at least some the indentations have a perforation therein.

14. A wound filling device according to claim 7 wherein the texturing comprise an array of repeated octagonal shapes.

15. A wound filling device according to claim 1 wherein the bag member is provided with a conduit port member attached thereto for the attachment of said at least one fluid carrying conduit.

16. A wound filling device according to claim 15 wherein the port member also provides passages therein for the connection of a wound cavity aspiration conduit.

17. A wound filling device according to claim 15 wherein the port member has a passage therein to co-operate with a further conduit to provide an air bleed and/or pressure reference connection.

18. A wound filling device according to claim 15 wherein the port member has a shroud member to prevent occlusion of an aspiration port therein by an overlying wound sealing membrane

19. (canceled)

20. A wound filling device according claim 1 further comprising: an aspiration conduit connected to aspiration means; and a sealing membrane for sealing said wound and defining a wound cavity.

21. Apparatus according to claim 20 further comprising an additional conduit to said wound cavity.

22. Apparatus according to claim 21 wherein said additional conduit is a bleed or reference pressure conduit.

23. (canceled)
